(19) United States
(12) Patent Application Publication
(10) Pub. No.: US 2026/0355101 A1
(43) Pub. Date: Nov. 12, 2026

(54) HYBRID NANOCOMPOSITE FUSED VEGAN EXOSOMES AND NIOSOMES HAVINGMULTIPLE BIOACTIVE SUBSTANCES

(71) Applicant: BioSpectrum, Inc., Yongin-si (KR)

(72) Inventors: Hye Soo WANG, Seoul (KR); Jun Bo Sim, Osan-Si (KR); Eun Ae Cho, Seoul (KR); Kyung Baeg Roh, Hwaseong-si (KR); Eun Sun Jung, Suwon-si (KR); Deck Hoon Park, Seongnam-si (KR)

(21) Appl. No.: 19/665,092

(22) Filed: May 1, 2026

(30) Foreign Application Priority Data
May 12, 2025 (KR) 10-2025-0061001

(51) Int. Cl. A61K 8/97; A61K 8/11; A61K 8/14; A61K 8/99; A61K 9/50; A61Q 19/00

(52) U.S. Cl. CPC A61K 8/97; A61K 8/11; A61K 8/14; A61K 8/99; A61K 9/5084; A61Q 19/007; A61K 2800/805; A61K 2800/82

(57) ABSTRACT

The present disclosure relates to a method for preparing a hybrid nanocomposite in which exosomes and niosomes are fused capable of stably and efficiently delivering various bioactive substances to the skin or tissues of a subject by utilizing the exosomes and the niosomes that are biocompatible and high-efficiency delivery systems, and to uses of the hybrid nanocomposite prepared by the method.

### CROSS-REFERENCE TO RELATED PATENT APPLICATION

This application claims the benefit of Korean Patent Application No. 10-2025-0061001, filed on May 12, 2025, in the Korean Intellectual Property Office, the disclosure of which is incorporated herein in its entirety by reference.

### TECHNICAL FIELD

The present disclosure relates to a method for preparing a hybrid nanocomposite in which exosomes and niosomes are fused capable of stably and efficiently delivering various bioactive substances to the skin or tissues of a subject by utilizing the exosomes and the niosomes that are biocompatible and high-efficiency delivery systems, and to uses of the hybrid nanocomposite prepared by the method.

### BACKGROUND

Conventional technologies for delivering bioactive substances have mainly employed a method of delivering active ingredients by utilizing liposomes, niosomes, nanoparticles, or exosomes as a single carrier. Among them, the exosomes are an extracellular vesicle composed of a phospholipid bilayer having a size of 50 to 200 nm and have been actively studied in the cosmetic and biomedical fields based on advantages such as intercellular signal transmission, biocompatibility, and permeability.

However, most conventional exosome-based technologies use exosomes extracted from animal-derived stem cells or human-derived cells, and such raw materials have limitations such as difficulties in obtaining vegan certification, ethical issues, immune responses, and possibility of pathogenic contamination.

Accordingly, Korean Patent No. 10-2450893 discloses a technology using exosomes derived from hydrangea leaves, and further to the technology, Korean Patent Publication No. 10-2025-0027604 discloses hybrid exosomes obtained by fusing hydrangea-derived exosomes with synthetic liposomes.

However, these conventional technologies simply use plant-derived exosomes or rely on a simple mixing or physical incubation method of the exosomes and a single ingredient (e.g., sapogenin, etc.), and thus have limitations in inducing complex bioactive effects. In particular, there is a lack of structural fusion technology capable of stably incorporating two or more multiple bioactive substances within a single system and, at the same time, improving skin and tissue penetration and bioavailability.

Meanwhile, recently, niosomes have a bilayer structure similar to that of liposomes, but exhibit excellent stability and cost efficiency based on a non-ionic surfactant, and superior skin permeability, and thus have attracted attention as an alternative system.

However, technologies for stably delivering various bioactive substances by simultaneously utilizing the niosomes and the exosomes has not yet been sufficiently developed.

Accordingly, the present inventors have developed a technology for structurally incorporating multiple bioactive substances such as nucleic acids, phytochemicals, and peptides and stably delivering the same into a subject by utilizing the niosomes and the exosomes, thereby completing the present disclosure.

### PRIOR ART LITERATURE

### Patent Document

(Patent Document 0001) Korean Patent Publication No. 10-2019-0038743 (published on Apr. 9, 2019)
(Patent Document 0002) Korean Patent Publication No. 10-2024-0011894 (published on Jan. 29, 2024)
(Patent Document 0003) Korean Patent Publication No. 10-2025-0027604 (published on Feb. 27, 2025)
(Patent Document 0004) Korean Patent No. 10-2450893 (published on Oct. 6, 2022)

### SUMMARY

### Technical Problem

An object of the present disclosure is to provide a safe and ethical nanocomposite based on vegan raw materials. Conventional exosomes are mostly derived from animal-derived stem cells or human-derived cells, and thus there are difficulties in obtaining vegan certification and concerns regarding ethical issues and immune responses. In contrast, an object of the present disclosure is to provide a safer and more ethical nanocomposite by using exosomes derived from plants, plant callus, or microorganisms (vegan exosomes), rather than from animals.

Another object of the present disclosure is to provide a nanocomposite that provides complex physiological effects. Since conventional technologies have been utilized by simply incubating or mixing only a single bioactive substance with exosomes, there is a limitation in inducing complex physiological effects (e.g., anti-inflammatory, antioxidant, regenerative effects, etc.). In contrast, an object of the present disclosure is to provide a nanocomposite that provides multiple effects by simultaneously incorporating multiple bioactive substances such as nucleic acids, phytochemicals, and peptides.

Still another object of the present disclosure is to provide a nanocomposite that overcomes instability and permeability limitation of using exosomes alone. The exosome itself has excellent biocompatibility, but has physical instability, difficulty in formulation, and limitation in permeability. In contrast, the present disclosure is directed to providing a nanocomposite having a dual protective structure and improved penetrability into the skin or tissues by incorporating with niosomes.

Still another object of the present disclosure is to provide a method for preparing the nanocomposite having the above-described usefulness.

Still another object of the present disclosure is to provide a composition comprising the nanocomposite having the above-described usefulness.

Yet another object of the present disclosure is to provide a method for providing physiological activity induced by the multiple bioactive substances inserted into the nanocomposite by administering the nanocomposite having the above-described usefulness to a subject.

### Technical Solution

As one aspect, the present disclosure provides a method for preparing a hybrid nanocomposite in which vegan exosomes and niosomes are fused.

As a specific aspect, the method for preparing the hybrid nanocomposite in which vegan exosomes and niosomes are fused of the present disclosure comprises:
(S10) preparing vegan exosomes;
(S20) inserting multiple bioactive substances into the vegan exosomes of step (S10);
(S30) preparing niosomes; and
(S40) fusing the vegan exosomes into which the multiple bioactive substances of step (S20) are inserted and the niosomes of step (S30) by ultrasonically treating the same.

Hereinafter, with reference to FIG. 2, the method for preparing the hybrid nanocomposite in which the vegan exosomes and the niosomes are fused according to the present disclosure will be described in detail.

Step (S10) is a step of preparing the vegan exosomes.

In the present disclosure, the exosome is an extracellular vesicle derived from plants or microorganisms, rather than an extracellular vesicle derived from animals, and the cells derived from the plants comprise undifferentiated cells, stem cells, callus, etc. Since the exosome is an extracellular vesicle not derived from animal cells, it may be referred to as a vegan exosome in the present specification.

The plant refers to both woody plants and herbaceous plants. Examples thereof comprise fruit trees such as apple trees, pear trees, persimmon trees, grapevines, and ginkgo trees; coniferous trees such as pine trees, Korean pine trees, and larch trees; broadleaf trees such as camellia trees, forsythia, magnolia trees, and cherry trees; annual herbaceous plants such as cosmos, moss rose, and soybean; biennial herbaceous plants such as barley, wheat, shepherd's purse, and Arabidopsis; and perennial herbaceous plants such as dandelion, balloon flower, and mugwort.

The microorganism comprises all microorganisms having cells such as eukaryotes or prokaryotes such as bacteria, fungi, and yeast.

The undifferentiated cell is a cell before differentiation and may also be used as a meaning comprising a stem cell and/or a callus.

The stem cell is a cell having an ability to differentiate into all types of cells or cells of a specific tissue in a plant, and examples thereof comprise apical meristem, cambium, stomatal lineage stem cells, etc., but is not necessarily limited thereto.

The callus may be a mass of undifferentiated amorphous parenchymal cells of a plant, and may be a callus isolated from plant tissues, a callus obtained by inducing a dedifferentiation process of a plant explant, or a callus proliferated by culturing the callus. The plant tissue may be a specific part or all parts of a plant, such as a flower, a leaf, or a stem, a branch, a fruit, a root, etc.

In a specific embodiment, the exosome may be an exosome derived from a microorganism of the genus Lactobacillus, a camellia leaf, a camellia callus, etc.

In the present disclosure, “preparation of exosomes” refers to isolating exosomes from cells derived from plants or microorganisms, excluding animal cells, and may comprise concentration for increasing the concentration of the isolated exosomes after the isolation.

The isolation of the exosomes from the cells derived from plants or microorganisms is not necessarily limited thereto, but may be performed by methods such as centrifugation, ultrasonic centrifugation, ultrafiltration, chromatography, etc. after homogenizing the cells (or cell culture solution).

The homogenization of the cells is a process for isolating exosomes present within the cells in addition to exosomes secreted by the cells, and the exosomes may be isolated by mechanically homogenizing and disrupting the cell wall and/or the cell membrane. The mechanical homogenization, disruption, and isolation may be performed, for example, by a homogenizer, a high-pressure homogenizer, an extracorporeal shock wave, an ultra-sonicator, etc. The disruption and isolation may be performed using a single apparatus or a plurality of apparatuses.

The concentration of the isolated exosomes refers to purifying the isolated exosomes to increase the concentration thereof, and may be performed by a method of performing cell homogenization two or more times or a tangential flow filtration (TFF) method, etc.

As a specific example, the isolation and concentration of the exosomes may be performed by primarily homogenizing plant- or microorganism-derived cells with the high-pressure homogenizer to isolate the exosomes, and then secondarily homogenizing the same with the ultra-sonicator.

As another specific example, the isolation and concentration of the exosomes may be performed by primarily homogenizing the plant- or microorganism-derived cells with the high-pressure homogenizer to isolate the exosomes, and then subjecting the same to tangential flow filtration.

Step (S20) is a step of inserting the multiple bioactive substances into the vegan exosomes prepared in step (S10).

In the present disclosure, the multiple bioactive substances refer to two or more bioactive substances to be inserted into the exosomes. The bioactive substances are substances that are present in living bodies such as plants, animals, microorganisms, etc. and affect various physiological activities. In addition, the bioactive substances may be substances chemically synthesized or produced by recombinant microorganisms so as to have chemical structures or constituent components identical to those of substances present in a living body by 80% or more, preferably 90% or more, while affecting physiological activity at levels equivalent to those of the substances present in the living body.

In the present disclosure, the insertion refers to that the bioactive substances are positioned inside the vegan exosomes or are attached to the surface of the exosomes or between membranes of the exosomes by a chemically covalent or non-covalent bond.

According to an embodiment of the present disclosure, the bioactive substances are not necessarily limited thereto, but may be one or more selected from the group consisting of proteins, peptides, extracellular matrices, nucleic acids, lipids, carbohydrates, drugs, and phytochemicals.

For example, the protein may comprise one or more selected from the group consisting of plasma proteins, hormones, cytokines, enzymes, antibodies, growth factors, transcriptional regulatory factors, blood coagulation factors, vaccines, clusters of differentiation, and receptor antagonists.

For example, the plasma protein may comprise one or more selected from the group consisting of albumin, globulin, and fibrinogen. More specifically, the albumin may comprise Bovine Serum Albumin (BSA).

For example, the hormone may comprise one or more selected from the group consisting of hepatic growth hormone, follicle-stimulating hormone, luteinizing hormone, luteinizing hormone-releasing hormone, adrenocortical hormone, and thyroid-stimulating hormone.

For example, the enzyme may comprise one or more selected from the group consisting of glucocerebrosidase, iduronate-2-sulfatase, alpha-galactosidase-A, agalsidasealpha, alpha-L-iduronidase, butyrylcholinesterase, chitinase, glutamate decarboxylase, imiglucerase, lipase, uricase, platelet-activating factor acetylhydrolase, neutral endopeptidase, and myeloperoxidase.

For example, the antibody may comprise one or more selected from the group consisting of monoclonal antibodies, polyclonal antibodies, and antibody fragments such as scFv, Fab, Fab′, F(ab′)2, and Fd.

For example, the growth factor may comprise one or more selected from the group consisting of platelet-derived growth factor, epithelial growth factor, epidermal growth factor, and nerve growth factor. Here, the nerve growth factor may be ciliary neurotrophic factor, axogenesis factor-1, brain-natriuretic peptide, netrin, neurophil inhibitor factor, neurotrophic factor, neuturin, etc.

For example, the transcriptional regulatory factor is a factor that binds to RNA polymerase so as to regulate transcription and may comprise a transcription complex that binds to a transcription pre-initiation complex or a promoter. According to an example, the transcription complex may be a TATA-binding protein that binds to a TATA box of DNA, and may specifically be TFIIA, TFIIB, TFIID, etc.

For example, the blood coagulation factor may be factor I (fibrinogen), factor II (prothrombin), factor V (proaccelerin or labile factor), factor VII (proconvertin), factor VIII (antihemophilic factor A), factor IX (antihemophilic factor B), factor X (Stuart-Prower factor), factor XI (plasma thromboplastin antecedent (PTA)), factor XIII (fibrin-stabilizing factor), etc.

For example, the vaccine is not particularly limited as long as it is a vaccine capable of treating a human body, and any such vaccine may be applied. According to an example, the vaccine may be a vaccine antigen derived from a virus.

For example, the cluster of differentiation may act as a ligand that activates a receptor. According to a specific example, the cluster of differentiation may be a CD marker applicable to stem cells, leukocytes, etc.

For example, the receptor antagonist may be a protein that acts on receptor molecules in vivo to inhibit or suppress functions of neurotransmitters, hormones, etc.

For example, the peptide may be growth hormone-releasing peptide, glucagon-like peptides (GLP-1, etc.), macrophage peptide, thrombin receptor-activating peptide, fibrin-binding peptide, pancreatic polypeptide, gastrin-releasing peptide, plant peptide, acetyl hexapeptide-8, oligopeptide-1 (EGF), or biomimetic peptides such as tripeptide-1. According to a specific example, the peptide may be one or more selected from peptides isolated from camellia flower extract, peptides isolated from wheat extract, peptides isolated from soybean extract, peptides isolated from oat extract, etc.

For example, the extracellular matrix may comprise one or more selected from the group consisting of collagen, gelatin, elastin, osteocalcin, fibrinogen, fibromodulin, tenascin, laminin, osteopontin, osteonectin, perlecan, versican, von Willebrand factor, and vitronectin.

For example, the nucleic acid may be DNA, RNA, an oligonucleotide, etc.

For example, the lipid may be sphingolipid, ceramide, cholesterol, cholesterol ester, triglyceride, phospholipid, and fatty acid, etc. According to a specific example, the lipid may be camellia oil.

For example, the carbohydrate may be sugars, oligosaccharides, polysaccharides, etc.

For example, the drug may comprise at least one selected from antibiotic agents, anticancer agents, anti-inflammatory agents, antiviral agents, and antibacterial agents. According to a specific example, the antibiotic agent may comprise one or more selected from the group consisting of tetracycline, minocycline, doxycycline, ofloxacin, levofloxacin, ciprofloxacin, clarithromycin, erythromycin, cefaclor, cefotaxime, imipenem, penicillin, gentamicin, streptomycin, and vancomycin, or derivatives thereof. According to a specific example, the anticancer agent may comprise one or more selected from the group consisting of methotrexate, carboplatin, Taxol, cisplatin, 5-fluorouracil, doxorubicin, etoposide, paclitaxel, camptothecin, and cytosine arabinoside, or derivatives thereof. According to a specific example, the anti-inflammatory agent may comprise one or more selected from the group consisting of indomethacin, ibuprofen, ketoprofen, piroxicam, flurbiprofen, and diclofenac, or derivatives thereof. According to a specific example, the antiviral agent may comprise at least one selected from acyclovir and ribavirin, or derivatives thereof. According to a specific example, the antibacterial agent may comprise at least one selected from ketoconazole, itraconazole, fluconazole, amphotericin-B, and griseofulvin, or derivatives thereof.

The phytochemical is a chemical substance produced by a plant and may comprise, for example, one or more selected from the group consisting of flavonoids, carotenoids, phenolic acids, glucosinolates, vitamin derivatives, saponins, alkaloids, and lignans. According to a specific example, the flavonoid may be one or more selected from quercetin, kaempferol, catechin, anthocyanin, etc. According to a specific example, the carotenoid may be at least one selected from the group consisting of beta-carotene, lycopene, lutein, zeaxanthin, etc. According to a specific example, the phenolic acid may be one or more selected from caffeic acid, ferulic acid, etc. According to a specific example, the vitamin derivative may be bakuchiol, tocopheryl acetate, etc. According to a specific example, saponin may be ginsenoside, madecassoside, titrated extract of Centella asiatica, soyasaponin, etc.

According to an embodiment of the present disclosure, exosome insertion of the multiple bioactive substances may be performed while homogenizing a mixture of the multiple bioactive substances and the exosomes using a homogenizer.

The homogenization may be performed at a pressure of 800 to 1200 bar, preferably 900 to 1100 bar, more preferably 950 to 1050 bar, still more preferably 980 to 1020 bar, even more preferably 990 to 1010 bar, and most preferably 1000 bar. When the homogenization is performed at less than 800 bar, an amount of the bioactive substances inserted into the exosomes is insufficient, and when the homogenization is performed at more than 1200 bar, there is a problem that the structure of the exosomes is destroyed.

The homogenization may be performed at 5 to 50 °C, preferably 10 to 40 °C, and more preferably 15 to 30 °C. When the homogenization is performed at less than 5 °C, membrane fluidity of the exosomes decreases, and thus an amount of the bioactive substances to be inserted is insufficient, and when the homogenization is performed at 50 °C or higher, there is a problem in that the structure of the bioactive substances to be inserted into the exosomes is modified, such that they are unable to function as the bioactive substances.

The homogenization may be performed by repeating one or more times, preferably two to four times, and more preferably three times, however, even when the homogenization is performed more than four times, the amount of the bioactive substances inserted into the exosomes is not increased, and thus it is uneconomical and inefficient.

Step (S30) is a step of preparing the niosomes.

In the present disclosure, the niosome refers to a non-ionic surfactant-based vesicle, which is a microparticle having a hydrophilic space formed therein and having a closed lipid bilayer membrane having hydrophobic properties on the outside.

The niosome may be formed by comprising a non-ionic surfactant, a stabilizer, and a solvent. In addition, the niosome may be formed by further comprising an auxiliary emulsifier in addition to the above-described ingredients.

In the present disclosure, the term “non-ionic surfactant” refers to a surfactant that is not dissociated into ions, and is also referred to as a non-ionic active agent or a non-ionic surface-active agent. The properties of the ionic surfactant, unlike those of an ionic surfactant, may freely mix with other surfactants or electrolytes. The non-ionic surfactant is harmless to the skin, and has characteristics such as thickening of a shampoo base, improving stability at low temperatures, and excellent cleansing action while generating less foam, and thus may be used for various purposes such as a dispersant, an emulsifier, a detergent, and a dyeing auxiliary.

The non-ionic surfactant may have a hydrophilic-lipophilic balance (HLB) of 11 to 18, specifically 13 to 16, but is not necessarily limited thereto, and a non-ionic surfactant having properties of an emulsifier (or emulsifying agent), more specifically a water-soluble emulsifier, may be used.

In addition, the non-ionic surfactant is preferably biodegradable.

Specifically, polyglyceryl esters, sorbitan esters, polyoxyethylene sorbitan fatty acid esters, polyoxyethylene alkyl ethers, poloxamers, or mixtures thereof may be used as the non-ionic surfactant, but is not limited thereto, and a non-ionic surfactant having similar properties may be used.

The polyglyceryl ester may be, for example, polyglyceryl-3 oleate, polyglyceryl-4 oleate, polyglyceryl-6 oleate, polyglyceryl-10 oleate, polyglyceryl-10 stearate, polyglyceryl-3 stearate, polyglyceryl-4 laurate, polyglyceryl-4 caprate, polyglyceryl-6 caprylate, etc., but the type thereof is not necessarily limited thereto, and an appropriate type capable of dissolving cholesterol in an aqueous solvent may be selected.

The sorbitan ester may be, for example, sorbitan monolaurate (or Span 20), sorbitan monostearate (or Span 60), sorbitan monooleate, etc., but the type thereof is not necessarily limited thereto, and an appropriate type capable of dissolving cholesterol in an aqueous solvent may be selected.

The polyoxyethylene sorbitan fatty acid ester may be, for example, polyoxyethylene (20) sorbitan monolaurate (Tween 20), polyoxyethylene (20) sorbitan monostearate (Tween 60), polyoxyethylene (20) sorbitan monooleate (Tween 80), etc., but the type thereof is not necessarily limited thereto, and an appropriate type capable of dissolving cholesterol in an aqueous solvent may be selected.

The polyoxyethylene alkyl ether may comprise, for example, polyoxyethylene (4) lauryl ether (Brij 30), polyoxyethylene (23) lauryl ether (Brij 35), polyoxyethylene (20) stearyl ether (Brij 78), etc., but the type thereof is not necessarily limited thereto, and an appropriate type capable of dissolving cholesterol in an aqueous solvent may be selected.

In the present disclosure, the stabilizer is added to form an inner phase of the niosome and to stabilize the niosome. As an example of such stabilizer, one or more selected from the group consisting of betaine, butylene glycol, polyhydric alcohol, organic acid, organic salts, complex of sugar and protein, and polyols comprising glycerin, propylene glycol, and dipropylene glycol may be used, and may more preferably comprise glycols such as 1,3-butylene glycol, propylene glycol, dipropylene glycol, and polyethylene glycol; polyhydric alcohols such as 1,2-hexanediol; glycerols such as glycerin, diglycerin, and polyglycerin; saccharides such as sorbitol, mannitol, sugar, starch syrup, and lactitol; water-soluble polymers such as guar gum, sodium chondroitin sulfate, sodium hyaluronate, sodium alginate, methyl cellulose, hydroxymethyl cellulose, carboxyvinyl polymer, polyvinyl alcohol, polyvinylpyrrolidone, alkylated carboxylic vinyl polymer, sodium polyacrylate, gum arabic, and carrageenan; salts such as sodium chloride, magnesium chloride, and sodium lactate, and the like, and among these, it is preferable to comprise glycerin.

The stabilizer may be comprised in an amount of 100 parts by weight, 200 parts by weight, 300 parts by weight, 400 parts by weight, 500 parts by weight, 600 parts by weight, 700 parts by weight, 800 parts by weight, 900 parts by weight, 1000 parts by weight, or parts by weight within a range between any two of the foregoing values, based on 100 parts by weight of the non-ionic surfactant. Preferably, the stabilizer may be comprised in an amount of 200 parts by weight, 300 parts by weight, 400 parts by weight, 500 parts by weight, or parts by weight within a range between any two of the foregoing values. For example, the range may be 200 to 500 parts by weight, 300 to 500 parts by weight, 300 to 400 parts by weight, etc. When an addition amount of the stabilizer is less than the 100 parts by weight, the inner phase of the niosomes is not properly formed and a stability of the niosomes is poor, and when the addition amount exceeds the 1000 parts by weight, there is a disadvantage in that a viscosity of the composition sharply increases, thereby making it difficult to subsequently fuse with the vegan exosomes.

In the present disclosure, the auxiliary emulsifier is a substance that assists the surfactant so that a hydrophilic substance and a hydrophobic substance are mixed in a solvent without being phase-separated, and is not limited in type, but may be higher alcohols (cetyl alcohol, stearyl alcohol, myristyl alcohol, behenyl alcohol, cetostearyl alcohol, cetearyl alcohol), fatty acid esters (isopropyl myristate, cetyl octanoate, oleyl oleate, isobutyl adipate, cetyl 2-ethylhexanoate, N-alkyl glycol monoisostearate, glyceryl stearate), or lecithin, and preferably comprises lecithin.

Lecithin relates to naturally occurring or synthetic lecithin that may be appropriately purified. Suitable lecithin comprises lecithin derived from eggs or soybeans, but is not limited thereto. More suitable lecithin comprises dihexanoyl-L-alpha-lecithin, dioctanoyl-L-alpha-lecithin, didecanoyl-L-alpha-lecithin, didodecanoyl-L-alpha-lecithin, ditetradecanoyl-L-alpha-lecithin, dihexadecanoyl-L-alpha-lecithin, dioctadecanoyl-L-alpha-lecithin, dioleoyl-L-alpha-lecithin, dilinoleoyl-L-alpha-lecithin, and alpha-palmitol, but is not limited thereto.

Lecithin is generally a mixture of diglycerides of fatty acids linked to choline ester of phosphoric acid, and may comprise different amounts of other compounds depending on a method of isolation. Generally, commercial lecithin is a mixture of acetone-insoluble phosphatides. Preferably, the lecithin is obtained from seeds comprising soybean and corn, most preferably soybean, using methods well known in the art. Lecithin obtained from soybean is referred to as soy lecithin in the present disclosure.

The auxiliary emulsifier may be comprised in an amount of 100 to 500 parts by weight, preferably 200 to 400 parts by weight, and more preferably 300 to 400 parts by weight, based on 100 parts by weight of the non-ionic surfactant. When a content of the auxiliary emulsifier is less than the above range, there is a problem in that the stability of the niosome is reduced, and when the content exceeds the above range, there is a problem in that feel of use of the topical preparation for skin and cosmetic composition is deteriorated.

The ingredients may be added to a solvent and stirred so as to be completely mixed. In this case, the solvent used is not limited in the present disclosure as long as it is a water-soluble solvent, and preferably comprises lower alcohols such as ethanol, phosphate buffered saline (PBS), physiological saline, or water.

The solvent may be comprised in an amount of 180 to 220 parts by weight, preferably 190 to 210 parts by weight, and more preferably 200 parts by weight, based on 100 parts by weight of the non-ionic surfactant.

In the present disclosure, the niosome may be prepared by mixing the non-ionic surfactant, the stabilizer, and the solvent, or additionally mixing the auxiliary emulsifier, and subjecting the mixture to high-pressure homogenization. For example, the niosome may be prepared by mixing the non-ionic surfactant, the stabilizer, and the solvent, and homogenizing and dispersing the mixture at 600 to 800 bar with a high-pressure homogenizer. For example, the niosome may be prepared by mixing the non-ionic surfactant, the stabilizer, the auxiliary emulsifier, and the solvent, and homogenizing and dispersing the mixture at 600 to 800 bar with a high-pressure homogenizer. In this case, temperature and time are not particularly limited, and may be homogenized by appropriately adjusting according to an ingredient content of the niosome. The niosome prepared as described above has superior membrane fluidity compared to a conventionally prepared niosome, and thus fusion with the above-described vegan exosomes is easily achieved, and the multiple bioactive substances inserted into the vegan exosomes may be easily inserted into and maintained in the fused hybrid nanocomposite.

In addition, the homogenization may be performed by repeating one or more times, preferably two to four times, so that the non-ionic surfactant, the stabilizer, and the solvent, or the additionally comprised auxiliary emulsifier may be uniformly homogenized and dispersed.

In the present disclosure, an average particle diameter of the niosome is not particularly limited, but the average particle diameter may satisfy 10 to 200 nm. Since the average particle diameter of the niosome affects an average particle diameter of a finally prepared hybrid nanocomposite, when the average particle diameter of the niosome is less than the above range, there is a problem in that an amount of the multiple bioactive substances, which are active ingredients inserted into the finally prepared hybrid nanocomposite, is too small, and when the average particle diameter of the niosome exceeds the above range, there is a problem in that a size of the finally prepared hybrid nanocomposite is large, thereby reducing skin permeability.

Step (S40) is a step of mixing the vegan exosomes into which the multiple bioactive substances of step (S20) are inserted with the niosomes prepared in step (S30), and ultrasonically treating the same to fuse the vegan exosomes and the niosomes to form the hybrid nanocomposite.

The mixing of the vegan exosomes and the niosomes may be performed at a particle number of 0.1 to 2:0.1 to 2, preferably a particle number of 0.8 to 1.2:0.8 to 1.2, and more preferably a particle number of 1:1. Outside the above range, there is a problem in that a fusion efficiency decreases because a content of either one of the vegan exosomes or the niosomes is high.

The fusion of the vegan exosomes and the niosomes may be performed by an ultrasonic treatment, and the ultrasonic treatment is a method that is easy and simple regardless of pH and temperature conditions and may increase fusion efficiency when compared to a fusion method by simple mixing and stirring of the vegan exosomes and the niosomes and a method of mixing and fusing cationic lipids and helper lipids with a mixture of the vegan exosomes and the niosomes. The efficiency of the fusion refers to a size, uniformity, and stability of the hybrid nanocomposite.

The ultrasonic treatment may be performed under conditions of 30 to 50 kHz, preferably 35 to 45 kHz, more preferably 38 to 42 kHz, and most preferably 40 kHz for 8 to 12 minutes, preferably 9 to 11 minutes, and more preferably 10 minutes.

The hybrid nanocomposite formed by such ultrasonic treatment forms a bilayer or a trilayer by irregularly arranging a portion of the bilayer constituting the vegan exosomes and a portion of the bilayer constituting the niosomes, and the multiple bioactive substances inserted into the vegan exosomes are inserted into the hybrid nanocomposite. In this case, the insertion refers to that the bioactive substances are positioned inside the hybrid nanocomposite or that the bioactive substances are attached to a surface of the hybrid nanocomposite or between membranes of the hybrid nanocomposite by a chemically covalent bond or non-covalent bond. Accordingly, in the present specification, the terms “hybrid nanocomposite” or “hybrid nanocomposite in which the exosomes and the niosomes are fused” is used as a meaning of comprising the insertion of the multiple bioactive substance.

In the present disclosure, the average particle diameter of the nanocomposite in which the vegan exosomes and the niosomes are fused is not particularly limited, but the average particle diameter may satisfy 100 to 200 nm. When the average particle diameter of the hybrid nanocomposite is less than the above range, there is a problem in that an amount of the multiple bioactive substances to be inserted is too small, and when the average particle diameter of the hybrid nanocomposite exceeds the above range, there is a problem in that, due to a large size thereof, in vivo permeation is difficult, thereby reducing in vivo delivery of the inserted multiple bioactive substances.

In the present disclosure, the hybrid nanocomposite in which the vegan exosomes and the niosomes are fused has a uniformity of 0.1 to 0.2, preferably 0.10 to 0.15, and more preferably 0.10 to 0.14, and thus has a uniform size.

In the present disclosure, the hybrid nanocomposite in which the vegan exosomes and the niosomes are fused has a zeta potential of −26.80 to −27.50, preferably a zeta potential of −27.00 to −27.50.

In the present disclosure, the hybrid nanocomposite in which the vegan exosomes and the niosomes are fused is sufficiently stable to be stored for a long period at 0 to 50 °C without changes in the average particle diameter and uniformity. According to a specific example, the hybrid nanocomposite of the present disclosure exhibited the same particle number, zeta potential, and average particle diameter as on day 0 of preparation even when stored at 0 to 50 °C for 4 weeks.

As another aspect, the present disclosure provides a hybrid nanocomposite in which the vegan exosomes and the niosomes are fused, prepared by the above-described method.

The hybrid nanocomposite in which the vegan exosomes and the niosomes are fused of the present disclosure forms a bilayer or a trilayer by irregularly arranging a portion of a bilayer constituting the vegan exosomes and a portion of a bilayer constituting the niosomes, and multiple bioactive substances to be delivered into a subject are present on a surface of the hybrid nanocomposite, between membranes of the hybrid nanocomposite, and inside the hybrid nanocomposite, and thus is a carrier that delivers the multiple bioactive substances in vivo.

In the present disclosure, the subject may be animals such as horses, cows, dogs, sheep, comprising humans, but is not necessarily limited thereto.

As described above, the multiple bioactive substances are two or more substances that are present in living bodies, such as plants, animals, or microorganisms, and affect various physiological activities, and the substances may comprise substances that are chemically synthesized or produced by recombinant microorganisms so as to have chemical structures or constituent components that are identical to those of substances present in the living bodies by 80% or more, preferably 90% or more, while affecting the physiological activities at levels equivalent to those of substances present in the living bodies.

The delivery may be performed by formulating into a pharmaceutical formulation, for example, a powder, granule, sustained-release granule, liquid, emulsion, suspension, spirit, tablet, sustained-release tablet, hard capsule, soft capsule, sustained-release capsule, pill, plaster, lotion, paste, spray, inhalant, patch, aerogel, topical preparation, etc., but is not necessarily limited thereto. The topical preparation may have a formulation such as a cream, gel, patch, spray, ointment, plaster, lotion, liniment, paste, or cataplasm.

In addition, the delivery may be performed by formulating into a cosmetic formulation, for example, in a form of a skin, emulsion, cream, sunscreen, foundation, essence, gel, pack, mask pack, foam cleanser, body cleanser, softening lotion, eyeliner, shampoo, rinse, soap, hair fixative, hair growth promoter, hair cream, hair styling gel, lubricant, toothpaste, aerosol, and transdermal patch, but is not necessarily limited thereto.

The hybrid nanocomposite in which the vegan exosomes and the niosomes are fused may have an average particle diameter of 100 nm, 110 nm, 120 nm, 130 nm, 140 nm, 150 nm, 160 nm, 170 nm, 180 nm, 190 nm, or 200 nm, or within a range between any two of the foregoing values. When the average particle diameter of the hybrid nanocomposite is less than 100 nm, there is a problem in that an amount of the multiple bioactive substances to be inserted is too small, and when the average particle diameter of the hybrid nanocomposite exceeds 200 nm, there is a problem in that, due to a large size thereof, in vivo permeation is difficult, thereby reducing in vivo delivery of the multiple bioactive substances inserted into the hybrid nanocomposite.

The hybrid nanocomposite in which the vegan exosomes and the niosomes are fused has a uniformity of 0.1 to 0.2, preferably 0.10 to 0.15, and more preferably 0.10 to 0.14, and thus has a uniform size.

In the present disclosure, the hybrid nanocomposite in which the vegan exosomes and the niosomes are fused has a zeta potential of −26.80 to −27.50, preferably a zeta potential of −27.00 to −27.50.

The hybrid nanocomposite in which the vegan exosomes and the niosomes are fused is sufficiently stable to be stored for a long period at 0 to 50 °C without changes in the average particle diameter and uniformity. According to a specific example, the hybrid nanocomposite of the present disclosure exhibited the same particle number, zeta potential, and average particle diameter as on day 0 of preparation even when stored at 0 to 50 °C for 4 weeks.

As still another aspect, the present disclosure provides a composition for delivering bioactive substances, comprising the hybrid nanocomposite in which the vegan exosomes and the niosomes are fused and into which the multiple bioactive substances are inserted.

In the delivery composition, all of the foregoing descriptions may be applied as they are to the hybrid nanocomposite and the multiple bioactive substances inserted therein.

The delivery is providing the multiple bioactive substances into the skin or tissues of a subject. Specifically, the delivery refers to providing the multiple bioactive substances into the skin or tissues of animals such as horses, cows, dogs, sheep, cats, and monkeys, comprising humans, in various formulations such as the above-described pharmaceutical formulation, the topical preparation formulation, the cosmetic formulation, or the food formulation. The pharmaceutical formulation, the topical preparation formulation, the cosmetic formulation, or the food formulation is as described in the present specification.

As an embodiment, when the multiple bioactive substances are PDRN, camellia oil, and a camellia flower-derived peptide, the multiple bioactive substances are delivered into a skin of a subject by a hybrid nanocomposite into which the multiple bioactive substances are inserted, thereby simultaneously exhibiting cell regeneration, antioxidant, anti-aging, wrinkle-improving, whitening, skin volume-enhancing, and moisturizing effects according to PDRN, the camellia oil, and the camellia flower-derived peptide.

As another embodiment, when the multiple bioactive substances are ceramide and tocopheryl acetate, cell barrier-strengthening, antioxidant, and anti-inflammatory effects according to the hybrid nanocomposite into which the multiple bioactive substances are inserted may be simultaneously exhibited.

As still another aspect, the present disclosure provides a composition comprising, as an active ingredient, a hybrid nanocomposite in which vegan exosomes and niosomes are fused and into which the multiple bioactive substances are inserted.

The composition exhibits effects according to type of the multiple bioactive substances inserted into the nanocomposite, which is an active ingredient. According to an embodiment, when the multiple bioactive substances are two or more antibiotic agents, a hybrid nanocomposite into which the two or more antibiotic agents are inserted constitutes a composition, and exhibits effects of the antibiotic agents in a subject according to administration of the composition. According to an embodiment, when the multiple bioactive substances are PDRN, camellia oil, and a camellia flower-derived peptide, a hybrid nanocomposite into which the multiple bioactive substances are inserted constitutes a composition and exhibits effects according to PDRN, the camellia oil, and the camellia flower-derived peptide in a subject according to administration of the composition.

The administration refers to introducing a predetermined substance into a subject by an appropriate method, and a route of administration of the composition may be any general route so long as it is capable of reaching a target tissue.

When the composition is a cosmetic composition, a pharmaceutical composition, or a topical composition for skin application, the nanocomposite in which the vegan exosomes and the niosomes are fused and into which the multiple bioactive substances that are active ingredients of the composition are inserted may be comprised in an amount of 0.0001 to 20 wt. %, preferably 0.0001 to 15 wt. %, more preferably 0.001 to 15 wt. %, and still more preferably 0.001 to 10 wt. %, based on a total wt. % of the composition. In addition, when the composition is a food composition, the hybrid nanocomposite in which the vegan exosomes and the niosomes are fused and into which the multiple bioactive substances that are active ingredients of the composition are inserted may be comprised in an amount of 0.0001 to 100 wt. %, preferably 0.0001 to 90 wt. %, more preferably 0.001 to 90 wt. %, and still more preferably 0.001 to 80 wt. %, based on a total weight of the composition.

As a specific aspect, the composition may be a cosmetic composition.

In addition to the hybrid nanocomposite in which the vegan exosomes and the niosomes are fused and into which the multiple bioactive substances are inserted, the cosmetic composition may further comprise ingredients commonly used in cosmetic compositions. For example, the cosmetic composition may comprise common adjuvants such as antioxidants, stabilizers, solubilizers, vitamins, pigments, and fragrances, and carriers.

The cosmetic composition may be prepared in any formulation that is commonly prepared in the art. For example, the cosmetic composition may be formulated into a solution, suspension, emulsion, paste, gel, cream, lotion, powder, soap, surfactant-containing cleansing, oil, powder foundation, emulsion foundation, wax foundation, or spray, etc. More specifically, the cosmetic composition may be prepared into a formulation such as a nourishing cream, astringent lotion, softening lotion, lotion, essence, nourishing gel, or massage cream, etc. However, the cosmetic composition is not particularly limited thereto.

When the formulation of the cosmetic composition is a paste, cream, or gel, animal oil, vegetable oil, wax, paraffin, starch, tragacanth, cellulose derivative, polyethylene glycol, silicone, bentonite, silica, talc, zinc oxide, etc., may be used as a carrier ingredient.

When the formulation of the cosmetic composition is a powder or spray, lactose, talc, silica, aluminum hydroxide, calcium silicate, or polyamide powder may be used as a carrier ingredient, and particularly when the formulation is a spray, it may additionally comprise a propellant such as chlorofluorohydrocarbon, propane/butane, or dimethyl ether.

When the formulation of the cosmetic composition is a solution or an emulsion, solvent, solubilizing agent, or emulsifying agent may be used as a carrier ingredient. For example, water, ethanol, isopropanol, ethyl carbonate, ethyl acetate, benzyl alcohol, benzyl benzoate, propylene glycol, 1,3-butylglycol oil, glycerol aliphatic esters, polyethylene glycol, or fatty acid esters of sorbitan may be used.

When the formulation of the cosmetic composition is a suspension, liquid diluents such as water, ethanol, or propylene glycol, suspending agents such as ethoxylated isostearyl alcohol, polyoxyethylene sorbitol ester, and polyoxyethylene sorbitan ester, and microcrystalline cellulose, aluminum metahydroxide, bentonite, agar, or tragacanth, etc. may be used as a carrier ingredient.

When the formulation of the cosmetic composition is a surfactant-containing cleansing, aliphatic alcohol sulfate, aliphatic alcohol ether sulfate, sulfosuccinic acid monoester, isethionate, imidazolinium derivative, methyltaurate, sarcosinate, fatty acid amide ether sulfate, alkylamidobetaine, aliphatic alcohol, fatty acid glyceride, fatty acid diethanolamide, vegetable oil, lanolin derivative, or ethoxylated glycerol fatty acid ester, etc. may be used as a carrier ingredient.

As a specific aspect, the composition may be a pharmaceutical composition. More specifically, the composition may be a pharmaceutical composition for preventing or treating skin diseases. The skin disease comprises one or more selected from the group consisting of atopic dermatitis, contact dermatitis, seborrheic dermatitis, psoriasis, acne, urticaria, allergic purpura, melasma, herpes zoster, herpes simplex, folliculitis, and skin cancer, but is not limited thereto.

The pharmaceutical composition refers to a composition prepared for a purpose of preventing or treating diseases, and may each be formulated and used in various forms according to a conventional method. For example, the pharmaceutical composition may be formulated into an oral formulation such as a powder, granule, tablet, capsule, suspension, emulsion, syrup, and aerosol, and may be used by formulating into a parenteral formulation using diluents or excipients such as lubricants, wetting agents, flavoring agents, emulsifiers, suspending agents, preservatives, surfactants, etc. In addition, the pharmaceutical composition may be formulated and used in a form of an injection, a topical preparation, or a topical preparation for skin.

In addition, depending on each formulation, the pharmaceutical composition may be prepared by further comprising a pharmaceutically acceptable carrier known in the art, for example, buffers, analgesic agents, solubilizing agents, isotonic agents, stabilizers, bases, etc.

As used in the present disclosure, the term “pharmaceutically acceptable carrier” may refer to a carrier or a diluent that does not irritate a living body and does not impair a biological activity and characteristics of an administered active ingredient. The type of the carrier usable in the present disclosure is not particularly limited, and any carrier may be used as long as it is commonly used in the relevant technical field and is pharmaceutically acceptable. Non-limiting examples of the carrier comprise saline, sterile water, Ringer solution, buffered saline, albumin injection solution, dextrose solution, maltodextrin solution, glycerol, ethanol, etc. These may be used alone or in a mixture of two or more types. The carrier may be a non-naturally occurring carrier.

The pharmaceutical composition may be administered in a pharmaceutically effective amount. The pharmaceutically effective amount refers to an amount sufficient for treating a disease with a reasonable benefit/risk ratio applicable to medical treatment and not so much as to cause side effects, and an effective dose level may be determined based on factors such as a patient's health condition, a type and severity of a disease, activity of a drug, sensitivity to a drug, a method of administration, time of administration, a route of administration, and excretion rate, duration of treatment, drugs used in combination or used concurrently, and other factors well known in the medical field. Specifically, the dose is generally 0.01 mg to 5000 mg per day per 1 kg of body weight of an administration subject, and may be administered once a day to several times a day in divided doses at predetermined time intervals according to a determination of a doctor or a pharmacist, but is not limited thereto.

As a specific aspect, the composition may be a topical composition for skin application.

The topical composition for skin application may be formulated by containing a cosmetically or dermatologically acceptable medium or base. The topical composition for skin application may be provided in any formulation suitable for topical application, for example, in a form of a solution, gel, solid, paste anhydrous product, emulsion obtained by dispersing an oil phase in an aqueous phase, suspension, microemulsion, microcapsule, microgranule, or ionic (liposome) and non-ionic vesicular dispersant, or in a form of a cream, skin, lotion, powder, ointment, spray, pack, or conceal stick. The topical composition for skin application may also be used in a form of a foam or in a form of an aerosol composition further containing a compressed propellant. These compositions may be prepared according to conventional methods in the art.

In addition, the topical composition for skin application may contain a fatty substance, organic solvent, solubilizing agent, thickener, gelling agent, softener, antioxidant, suspending agent, stabilizer, foaming agent, fragrance, surfactant, water, ionic or non-ionic emulsifier, filler, metal ion sequestering agent, chelating agent, preservative, vitamin, blocking agent, wetting agent, essential oil, dye, pigment, hydrophilic or lipophilic active agent, lipid vesicle, or adjuvant commonly used in the cosmetic or dermatological field, such as any other ingredients commonly used in cosmetics. The adjuvant is introduced in an amount generally used in the cosmetic or dermatological field.

As a specific aspect, the composition may be a food composition.

The food composition may comprise all foods in the ordinary sense, and may be used interchangeably with terms known in the art, such as functional food, health functional food, etc.

When the food composition is used as a food additive, the hybrid nanocomposite in which the vegan exosomes and the niosomes are fused and into which the multiple bioactive substances are inserted may be added as it is or may be used together with other foods or food ingredients, and may be appropriately used according to conventional methods. A mixing amount of the active ingredient may be suitably determined according to an intended use (prevention, health, or therapeutic treatment), and may further comprise sitologically acceptable food supplement additives. Since the composition of the present disclosure is not only a substance derived from a natural product but also has been confirmed to be safe from human safety tests, there is no significant limitation on the mixing amount.

As yet another aspect, the present disclosure relates to a method for providing physiological activity induced by multiple bioactive substances by administering the hybrid nanocomposite in which the vegan exosomes and the niosomes are fused and into which the multiple bioactive substances are inserted in a subject to deliver the multiple bioactive substances into the subject.

All of the foregoing descriptions may be applied as they are to the hybrid nanocomposite, the multiple bioactive substances inserted therein, and the physiological activity induced by the multiple bioactive substances.

As described above, the subject may be a horse, a cow, a dog, a sheep, a cat, or a monkey, comprising a human, but is not limited thereto.

The administration refers to introducing a predetermined substance into a subject by an appropriate method, and a route of administration of the composition may be any general route so long as it is capable of reaching a target tissue. For example, the administration may be intraperitoneal administration, intravenous administration, intramuscular administration, subcutaneous administration, intradermal administration, oral administration, topical administration, intranasal administration, pulmonary administration, or rectal administration, but is not limited thereto. Preferably, the administration may be subcutaneous administration, intradermal administration, or topical administration.

### Advantageous Effects

The hybrid nanocomposite in which vegan exosomes and niosomes are fused according to the present disclosure is a biocompatible carrier that overcomes ethical and immunological limitations of conventional animal-derived exosome-based technologies, is capable of effectively delivering complex bioactive effects as one or more bioactive substances are stably inserted therein, and is a novel carrier capable of maximizing, through fusion of the exosomes and the niosomes, not only permeability and stability, but also a sustained-release effect. Accordingly, the hybrid nanocomposite can be usefully used in various formulations in cosmetics, pharmaceuticals, topical preparations, foods, etc. as a carrier of the multiple bioactive substances.

### BRIEF DESCRIPTION OF THE DRAWINGS

FIG. 1 is a view schematically illustrating a structure of a hybrid nanocomposite in which vegan exosomes and niosomes are fused and into which multiple bioactive substances are inserted.

FIG. 2 is a view illustrating a method for preparing a hybrid nanocomposite in which vegan exosomes and niosomes are fused and having multiple bioactive substances according to an embodiment of the present disclosure.

FIG. 3 is a view of measuring particle sizes of each of exosomes of Preparation Example 2, niosomes of Example 4, and a hybrid nanocomposite of Preparation Example 5 by Dynamic Light Scattering (DLS) and comparing the same according to an embodiment of the present disclosure.

FIG. 4 is a result of electrophoresis confirming that a bioactive substance is inserted into a hybrid nanocomposite in which vegan exosomes and niosomes are fused, prepared according to an embodiment of the present disclosure.

FIG. 5 is a fluorescence image analysis result confirming that the hybrid nanocomposite prepared according to an embodiment of the present disclosure is a complex formed by fusion of vegan exosomes and niosomes.

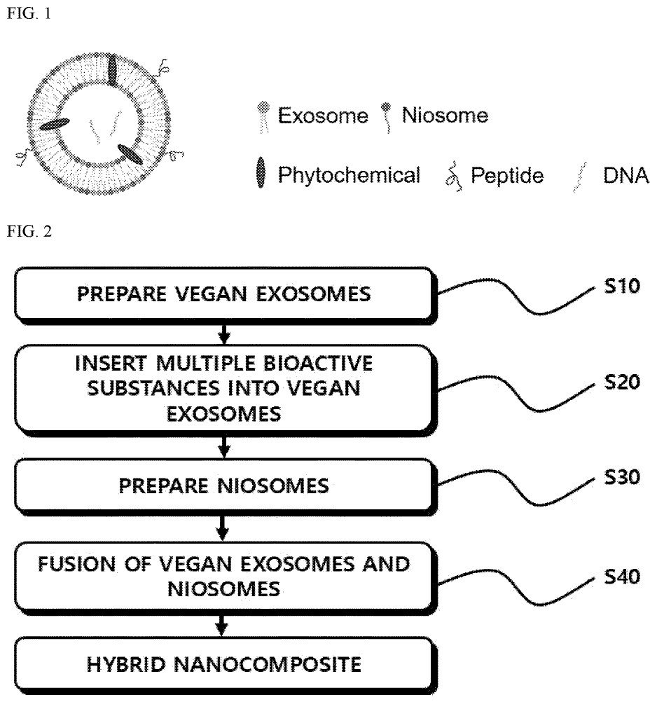

### DETAILED DESCRIPTION OF THE EMBODIMENTS

Hereinafter, Examples of the present disclosure will be described in detail to help understanding of the present disclosure. However, various modifications and changes may be made to Examples of the present disclosure, and it should be understood that the scope of the disclosure is not limited to following Examples. Examples of the present disclosure are provided to those having ordinary skill in the art for more complete description of the present disclosure.

### Example 1: Extraction and Purification of Vegan Exosomes

Lactobacillus rhamnosus BS Pro-8 (Accession number: KCTC 15876 BP) was cultured in a modified MRS medium under anaerobic conditions at 37 °C for 3 days. The culture medium after completion of culturing was centrifuged at 5000 rpm for 30 minutes and filtered through a 0.22 μm bottle-top filter to separate and remove bacterial cells, cell debris, etc., and a supernatant was subjected to 100 kDa size cut-off (MWCO) (PES membrane, Merck) through TFF to purify and concentrate high-purity exosomes. As a result of confirming sizes of the exosomes by Dynamic Light Scattering (DLS, ELSZ-2000ZS, Otsuka Electronics), an average size of the exosomes was about 187.7 nm.

### Example 2: Preparation of a Bioactive Substance

Polydeoxyribonucleotide (PDRN), camellia oil, and a plant-derived peptide were prepared as the bioactive substance. Specifically, high-purity (>90%) PDRN purchased from HTL was used as PDRN. Camellia oil was prepared by extracting camellia oil from camellia seeds by a cold-press method and then refining the camellia oil using activated carbon and bentonite. The peptide was obtained in a form of a crude peptide by softening camellia flowers in hot water (80 °C, 3 hours) and then treating the softened camellia flowers with an enzyme (1% protamax of substrate, 50 °C, 6 hours). As a result of measuring using MALDI-TOF MS (MALDI TOF Voyager DE-STR, Applied Biosystems, USA), a molecular weight of the peptide was confirmed to be about 1,200 Da.

### Example 3: Preparation of Vegan Exosomes into which Multiple Bioactive Substances are Inserted

PDRN, the camellia oil, and the peptide of Example 2 were mixed with the purified exosomes of Example 1, and PDRN, the camellia oil, and the peptide were inserted into the exosomes through a high-pressure homogenizer under the conditions shown in Table 1. Reaction conditions are shown in Table 1, and the exosomes prepared according to the reaction conditions were designated as Preparation Examples 1 to 3. Through this process, exosomes into which PDRN, the camellia oil, and the peptide are inserted were prepared.

TABLE 1

| Category | Homogenizer Pressure and Cycle |
|---|---|
| Preparation Example 1 | 600 bar, 3 cycle |
| Preparation Example 2 | 1000 bar, 3 cycle |
| Preparation Example 3 | 1400 bar, 3 cycle |

### Example 4: Preparation of Niosomes

100 parts by weight of a biodegradable non-ionic surfactant of a plant-based polyglyceryl ester series, 400 parts by weight of glycerin, and 200 parts by weight of water were mixed, and passed through a high-pressure homogenizer for 2 cycles at a pressure of 600 to 800 bar to prepare nanosized niosomes.

### Example 5: Preparation of a Hybrid Nanocomposite in which Vegan Exosomes and Niosomes are Fused and into which Multiple Bioactive Substances are Inserted

The vegan exosomes into which the multiple bioactive substances are inserted, prepared through Preparation Examples 1 to 3, were added to the niosomes prepared in Example 4 at a particle number of 1:1, and then subjected to an ultrasonic (Sonics vibracell, 40 kHz, 10 minutes) membrane fusion reaction to prepare a hybrid nanocomposite fused with the niosomes. Reaction conditions are shown in Table 2, and a structure thereof is schematically illustrated in FIG. 1.

TABLE 2

| Category | Exosome + Niosome Hybrid Conditions |
|---|---|
| Preparation Example 4 | Preparation Example 1 + Example 4 |
| Preparation Example 5 | Preparation Example 2 + Example 4 |
| Preparation Example 6 | Preparation Example 3 + Example 4 |

### Example 6: Measurement of a Hybrid Nanocomposite in which Vegan Exosomes and Niosomes are Fused

In order to confirm stability under each condition of the hybrid nanocomposites of Preparation Examples 4 to 6 prepared in Example 5, particle numbers were measured using Nanoparticle Tracking Analysis (NTA), and particle sizes were measured using DLS, and the result is shown in Table 3. The vegan exosomes of Preparation Example 2 were designated as a comparative example.

Preparation Example 2 had a size of 155.9 nm, and a PI value was 0.141. Preparation Example 4 had a size of 183.5 nm, and a PI value was 0.431. Preparation Example 5 had a size of 162.7 nm, and a PI value was 0.125. Preparation Example 6 had a size of 32.3 nm, and a PI value was 0.518. In a case of Preparation Example 6, it was confirmed that all of the exosomes were disrupted by a high-pressure environment and failed to be incorporated with the niosomes, such that an average particle size and particle number were not formed.

TABLE 3

| | Preparation | Preparation | Preparation | Preparation |
|---|---|---|---|---|
| Category | Example 2 | Example 4 | Example 5 | Example 6 |
| DLS (nm) | 155.9 | 183.5 | 162.7 | 32.3 |
| PI | 0.141 | 0.431 | 0.125 | 0.518 |
| Particle | 9.7E+10 | 1.25E+11 | 1.7E+11 | 4.5E+08 |
| Number | | | | |
| (particles/mL) | | | | |

### Example 7: Stability Experiment of a Hybrid Nanocomposite in which Vegan Exosomes and Niosomes are Fused

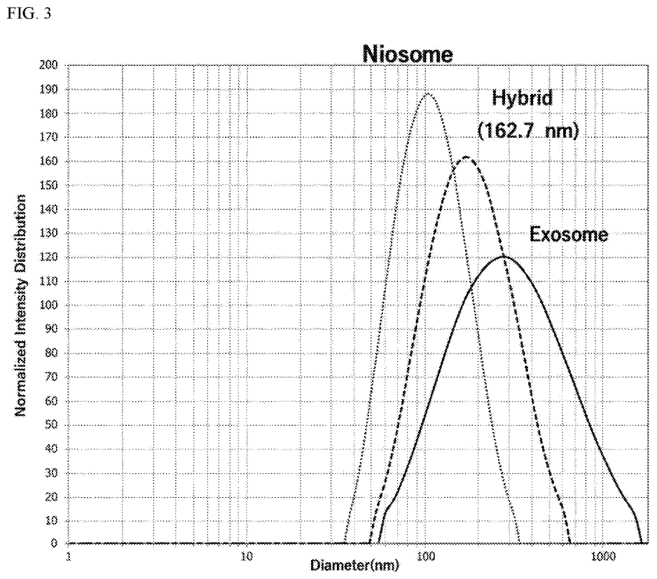

In order to confirm stability according to a storage period and temperature of the hybrid nanocomposites of Preparation Examples 4 to 6 prepared in Example 5, particle numbers and zeta potentials of the hybrid nanocomposites of Preparation Examples 4, 5, and 6 were measured using NTA and are shown in Tables 4 and 5, and particle sizes were measured using DLS and are shown in Table 6 and FIG. 3.

TABLE 4

| Particle | | | | | | | | | |
|---|---|---|---|---|---|---|---|---|---|
| Number | Preparation Example 4 | | | Preparation Example 5 | | | Preparation Example 6 | | |
| (particle/ml) | 4 °C | 25 °C | 45 °C | 4 °C | 25 °C | 45 °C | 4 °C | 25 °C | 45 °C |
| Day 0 | 1.25E+11 | | | 1.7E+11 | | | 4.5E+08 | | |
| Week 4 | 9.8E+11 | 3.1E+12 | 5.5E+12 | 1.7E+12 | 1.5E+12 | 1.9E+12 | 4E+08 | 3.8E+08 | 6.7E+08 |

TABLE 5

| Zeta | | | | | | | | | |
|---|---|---|---|---|---|---|---|---|---|
| Potential | Preparation Example 4 | | | Preparation Example 5 | | | Preparation Example 6 | | |
| (mV) | 4 °C | 25 °C | 45 °C | 4 °C | 25 °C | 45 °C | 4 °C | 25 °C | 45 °C |
| Day 0 | −25.99 | | | −27.27 | | | −26.00 | | |
| Week 4 | −26.00 | −25.45 | −26.01 | −26.50 | −29.78 | −25.88 | −26.24 | −26.48 | −26.83 |

TABLE 6

| Particle | Preparation | | | Preparation | | | Preparation | | |
|---|---|---|---|---|---|---|---|---|---|
| Size | Example 4 | | | Example 5 | | | Example 6 | | |
| (nm) | 4 °C | 25 °C | 45 °C | 4 °C | 25 °C | 45 °C | 4 °C | 25 °C | 45 °C |
| Day 0 | 163.9 | | | 146.6 | | | 35.7 | | |
| Week 4 | 351.5 | 305.8 | 399.8 | 167.5 | 162.4 | 159.4 | 45.1 | 32.5 | 40.8 |

In a case of Preparation Example 4, it was confirmed that separation occurred over time, resulting in changes in the particle number and the particle size. In contrast, in a case of Preparation Example 5, it was confirmed that a stable structure was maintained.

It was confirmed that when the pressure is too low as in Preparation Example 4, homogenization is not achieved, resulting in instability and failure of proper insertion of the bioactive substance, whereas, when the pressure is too high as in Preparation Example 6, the exosomes are disrupted and failed to properly fuse with the niosomes. Through this, it was confirmed that the hybrid nanocomposite prepared under the conditions of Preparation Example 5 was the most stable.

### Example 8: Experiment for Confirming Safety of a Hybrid Nanocomposite for Human Skin

### 8-1. Preparation of a Topical Preparation for Skin Comprising a Hybrid Nanocomposite

In order to confirm whether the hybrid nanocomposite in which the vegan exosomes and the niosomes are fused according to the present disclosure is safe for human skin, a topical preparation for skin comprising the nanocomposite of Preparation Example 5 was prepared with ingredients and contents shown in Table 7 below, and a skin safety verification experiment using the same was performed.

For preparation of the topical preparation for skin, purified water, glycerin, and butylene glycol were mixed and dissolved at 70 °C (aqueous phase part), and remaining ingredients other than the three ingredients and trimethanolamine were dissolved at 70 °C (oil phase part). The oil phase part was added to the aqueous phase part and stirred with a homomixer (Tokushu Kika, Japan) to perform primary emulsification, and trimethanolamine was finally added thereto. After removing bubbles generated in the mixed solution, it was cooled to room temperature to prepare the topical preparation for skin.

TABLE 7

| | Content (%) | |
|---|---|---|
| Ingredient | Control Group | Test Group |
| Purified water | 72.0 | 67 |
| Glycerin | 8.0 | 8.0 |
| Butylene glycol | 4.0 | 4.0 |
| Hybrid nanocomposite in which vegan | 0 | 5.0 |
| exosomes and niosomes are fused | | |
| Caprylic capryl triglyceride | 8.0 | 8.0 |
| Squalane | 5.0 | 5.0 |
| Cetearyl glucoside | 1.5 | 1.5 |
| Sorbitan stearate | 0.4 | 0.4 |
| Cetearyl alcohol | 1.0 | 1.0 |
| Trimethanolamine | 0.1 | 0.1 |
| Total weight | 100 | 100 |

### 8-2. Primary Skin Irritation Test

Whether the hybrid nanocomposite in which the vegan exosomes and the niosomes are fused of Preparation Example 5 causes irritation on the skin was measured by performing a 24-hour occlusive patch on the back of 30 healthy adults using each topical preparation for skin prepared in Example 8-1.

For the patch method, a chamber (IQ Ultimate™) of Chemotechnique MB Diagnostics AB was used. 20 μL of each topical preparation for skin was placed in the chamber and the occlusive patch was performed for 24 hours. A skin reaction was observed 30 minutes and 24 hours after removal of the patch, and the presence and degree of skin irritation were visually evaluated according to evaluation criteria, and after an average skin reactivity was calculated according to determination criteria of Table 8 on the basis of Frosch & Kligman (1979) and CTFA guideline (2007), skin irritation of each topical preparation for skin was evaluated using following Experimental Formula 1 on the basis of a skin reactivity classified by applying the Draize method.

Formula for calculating an average skin irritation index

Σ[(Grade×number of test subjects exhibiting reaction)/(4; highest grade×total number of test subjects)]÷number of evaluations [Experimental Formula 1]

TABLE 8

| Symbol | Grade | Irritation Evaluation |
|---|---|---|
| − | 0 | No reaction |
| + | 1 | Slight but clearly visible erythema |
| ++ | 2 | Clearly visible erythema |
| | | accompanied by papules or edema |
| +++ | 3 | Severe erythema accompanied |
| | | by edema and papules |
| ++++ | 4 | Severe erythema accompanied |
| | | by edema and blisters |

TABLE 9

| Skin Reactivity | Assessment of Skin Irritation |
|---|---|
| 0.00-0.25 | Non-irritating |
| 0.26-1.00 | Slightly irritating |
| 1.01-2.50 | Moderately irritating |
| 2.51-4.00 | Severely irritating |

TABLE 10

| | Number of Test Subjects Exhibiting Reaction | | | | | | | | |
|---|---|---|---|---|---|---|---|---|---|
| | After 30 minutes | | | | After 24 hours | | | | Average |
| Test Substance | 1st | 2nd | 3rd | 4th | 1st | 2nd | 3rd | 4th | Reactivity |
| Control Group | — | — | — | — | — | — | — | — | 0.00 |
| Test Group | — | — | — | — | — | 1 | — | — | 0.00 |
| Number of Test | 30 | 30 | 30 | 30 | 30 | 30 | 30 | 30 | |
| Subjects | | | | | | | | | |

In Table 10, no irritation reaction was exhibited in both the control group and the test group. When calculated according to the above-described formula, an average reactivity was 0.00, and thus the topical preparation for skin comprising the nanocomposite in which the vegan exosomes and the niosomes are fused according to the present disclosure was determined to be a non-irritating substance and a substance safe for human skin, similar to the topical preparation for skin of the control group.

### Example 9: Preparation of a Hybrid Nanocomposite in which Plant-Derived Vegan Exosomes and Niosomes are Fused

### 9-1. Isolation and Concentration of Plant-Derived Vegan Exosomes

Water was added to Centella asiatica leaves at a weight ratio of 10-fold and finely ground using a mixer to perform primary disruption, and then homogenization was performed under conditions of 7000 rpm and 30 minutes to perform secondary disruption that disrupted cell walls. After removing other impurities through centrifugation and a 0.22 μm filter, a supernatant was subjected to 100 kDa size cut-off (MWCO) (PES membrane, Merck) through a Tangential flow filtration (TFF) apparatus to purify and concentrate high-purity exosomes. As a result of confirming sizes of the exosomes by DLS, an average size of the exosomes was about 177.7 nm.

9-2. Preparation of Vegan Exosomes into which Multiple Bioactive Substances are Inserted

Plant-derived vegan exosomes into which multiple bioactive substances comprising PDRN, camellia oil, and a peptide are inserted were prepared in the same manner as in Example 3.

9-3. Preparation of a Hybrid Nanocomposite in which Plant-Derived Vegan Exosomes and Niosomes are Fused

In the same manner as in Example 5, the plant-derived vegan exosomes into which the multiple bioactive substances are inserted, prepared in Example 9-2, were added to the niosomes prepared in Example 4 at a particle number of 1:1, and then subjected to an ultrasonic (Sonics vibracell, 40 kHz, 10 min) membrane fusion reaction to prepare a hybrid nanocomposite in which the plant-derived vegan exosomes and the niosomes are fused.

### Example 10: Preparation of a Hybrid Nanocomposite in which Plant Callus-Derived Vegan Exosomes and Niosomes are Fused

### 10-1. Culture of Callus

Camellia petals cut into an appropriate size were sterilized by immersing the petals in 10% (v/v) hydrogen peroxide for 10 minutes and in 70% (v/v) ethanol for 1 minute, and then shaking the petals in a 2% (v/v) sodium hypochlorite solution for 30 minutes. Callus was cultured by washing sterilized camellia tissue with sterile water five or more times, then finely cutting into pieces of about 1 cm, and then inoculating on a Murashinge and Skoog (MS, 1962) medium containing sucrose, Gelrite, and growth regulators (Indole-3-acetic acid, IAA; Zeatin), followed by culturing at 25 °C for 8 weeks. The cultured callus was subcultured again at 5-week intervals to proliferate and acclimate the same, and the stably proliferated callus was finally obtained after suspension-culturing again in an air-lift bioreactor under dark conditions at 25 °C for 6 weeks.

### 10-2. Isolation and Concentration of Plant Callus-Derived Vegan Exosomes

The callus culture solution prepared in Example 10-1 was homogenized using a high-pressure homogenizer (Scientz-150, Ningbo Scientz Biotechnology Co., Ltd). Specifically, the callus culture solution was homogenized by repeating one to three times, a process of homogenizing in a microfluidizer (MF) at a pressure of 2,000 bar for 20 minutes and then allowing the same to stand at room temperature for 10 minutes. After homogenization using MF, efficiency under each homogenization condition was confirmed by additionally performing a homogenization process for 10 minutes (30 sec pulse on, 30 sec pulse off, 10 cycles) under a 20% power condition using a 500 W, 20 kHz ultra-sonicator (VC505, Sonics & Materials, Inc.).

The homogenized callus was centrifuged at 10,000×g for 30 minutes at room temperature and collected a supernatant to primarily remove solids, and the collected supernatant was filtered through a 0.22 μm sterilizing filter to obtain an exosome solution.

The exosome solution obtained through filtration was subjected to isolation and concentration of the exosomes using a Tangential Flow Filtration (TFF) apparatus. Concentration of the exosomes using TFF was performed with a 100 kDa molecular weight cut-off (MWCO) filter (Pellicon® 2 Mini Ultrafiltration Module Biomax, Millipore) and concentrated to 1/10 of a total volume of the exosome solution.

10-3. Preparation of Vegan Exosomes into which Multiple Bioactive Substances are Inserted

Plant callus-derived vegan exosomes into which multiple bioactive substances comprising PDRN, camellia oil, and a peptide are inserted were prepared in the same manner as in Example 3.

10-4. Preparation of a Hybrid Nanocomposite in which Plant Callus-Derived Vegan Exosomes and Niosomes are Fused

In the same manner as in Example 5, the plant callus-derived vegan exosomes into which the multiple bioactive substances are inserted, prepared in Example 10-3, were added to the niosomes prepared in Example 4 at a particle number of 1:1, and then subjected to an ultrasonic (Sonics vibracell, 40 kHz, 10 min) membrane fusion reaction to prepare a hybrid nanocomposite in which the plant callus-derived vegan exosomes and the niosomes are fused.

### Example 11: Confirmation of a Bioactive Substance Inserted into Hybrid Nanocomposite in which Vegan Exosomes and Niosomes are Fused

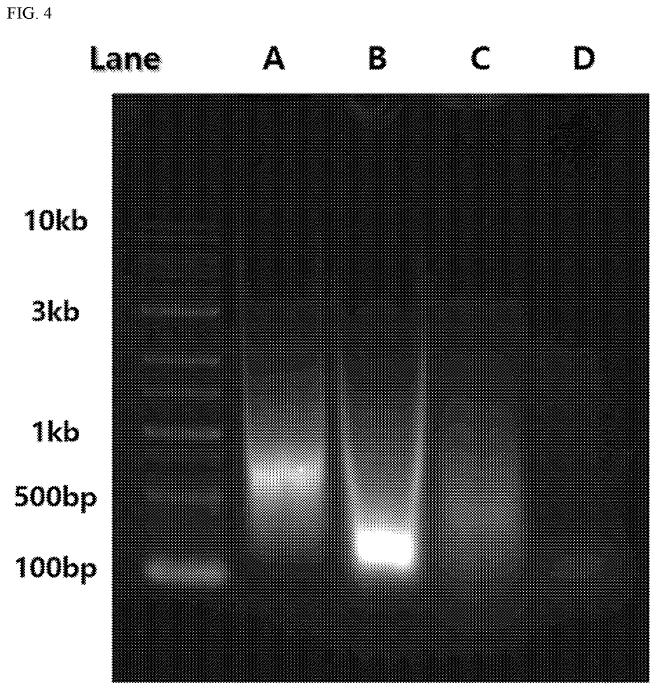

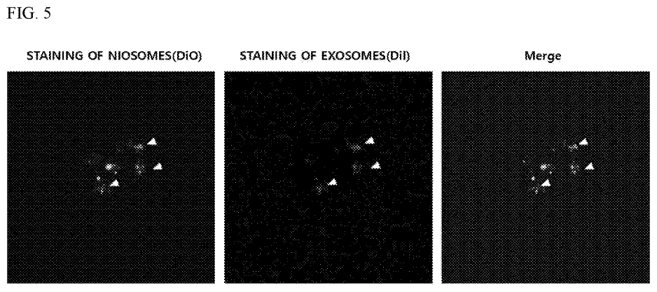

It was confirmed whether PDRN that is a bioactive substance was properly inserted into the hybrid nanocomposite in which the vegan exosomes and the niosomes are fused, prepared in Example 5. To this end, the hybrid nanocomposite in which the vegan exosomes and the niosomes are fused of Preparation Example 5 was treated with deoxyribonuclease (DNase) and ribonuclease (RNase) to remove uninserted PDRN and nucleic acids. Specifically, the hybrid nanocomposite in which the vegan exosomes and the niosomes are fused was treated with 0.1 mg/mL DNase and 50 μg/mL RNase, then reacted at 37 °C for 30 minutes to hydrolyze and remove PDRN not inserted into the exosomes, PDRN was confirmed by electrophoresis, and a result thereof is shown in FIG. 4. In the electrophoresis, lane A was loaded with PDRN before insertion into the hybrid complex, and lane D was loaded with PDRN treated with DNase and RNase. In the preparation process, the hybrid complex in which the vegan exosomes and the niosomes are fused was loaded into lane B, and the hybrid complex from which PDRN not inserted into the hybrid complex was removed by treatment with DNase and RNase was loaded into lane C.

As a result of the electrophoresis, a PDRN band was identified in lane A, and it was confirmed that PDRN was degraded by RNase and DNase in lane D. In lane B, both PDRN inserted into the hybrid complex and uninserted PDRN in a free state were observed. In lane C, PDRN in a free state was removed by RNase and DNase treatment, and only PDRN inserted into the hybrid complex was observed. Through this, it was confirmed that PDRN was successfully inserted into the hybrid complex and the complex was properly formed.

### Example 12: Fluorescence Microscopy Measurement of a Hybrid Nanocomposite in which Vegan Exosomes and Niosomes are Fused

It was confirmed whether the hybrid nanocomposite in which the vegan exosomes and the niosomes are fused, prepared in Example 5, was properly prepared. To this end, a lipid bilayer of the vegan exosomes of Example 3 was stained using DiI dye (Ex/Em-553/570) which is red fluorescence, and a lipid bilayer of the niosomes of Example 4 was stained using DiO (Ex/Em-484/501) which is green fluorescence. Each of the fluorescence-stained vegan exosomes and niosomes were used to prepare a hybrid nanocomposite by using the same method as in Example 5. Images of the prepared hybrid nanocomposite in which the vegan exosomes and the niosomes are fused were photographed at respective fluorescence wavelengths using a confocal microscope, and the fluorescence images were merged to determine hybridization of the vegan exosomes and the niosomes. A result thereof is shown in FIG. 5.

As shown in FIG. 5, it was confirmed that both the red fluorescence of the vegan exosomes and the green fluorescence of the niosomes were analyzed in the hybrid nanocomposite, and when these two types of fluorescence were merged, yellow fluorescence in which the red and green are combined in the same structure was analyzed. Accordingly, it was confirmed that the hybrid nanocomposite in which the vegan exosomes and the niosomes are fused, prepared through Example 5, was prepared.

A composition comprising the hybrid complex comprising the multiple bioactive substances of the present disclosure, wherein the hybrid complex is a hybrid complex in which the vegan exosomes and the niosomes are fused, may be prepared by following Formulation Examples, but is not limited thereto.

### Formulation Example 1: Cosmetic Formulation

### Formulation Example 1-1: Softening Lotion

A softening lotion comprising a hybrid nanocomposite in which vegan exosomes and niosomes are fused according to the present disclosure as an active ingredient was prepared according to a conventional method as in Table 11 below.

TABLE 11

| Ingredient | Wt. % |
|---|---|
| Hybrid nanocomposite in which vegan exosomes and | 5.0 |
| niosomes are fused | |
| Glycerin | 3.0 |
| Butylene glycol | 2.0 |
| Propylene glycol | 2.0 |
| Carboxyvinyl polymer | 0.1 |
| Ethanol | 10.0 |
| Triethanolamine | 0.1 |
| Preservatives, trace amounts of colorants, trace amounts of | 77.8 |
| fragrances, and trace amounts of purified water | |
| Total | 100.0 |

### Formulation Example 1-2: Nourishing Lotion

A nourishing lotion comprising a hybrid nanocomposite in which vegan exosomes and niosomes are fused according to the present disclosure as an active ingredient was prepared according to a conventional method as in Table 12 below.

TABLE 12

| Ingredient | Wt. % |
|---|---|
| Hybrid nanocomposite in which vegan exosomes and | 5.0 |
| niosomes are fused | |
| Beeswax | 4.0 |
| Polysorbate 60 | 1.5 |
| Sorbitan sesquioleate | 0.5 |
| Liquid paraffin | 5.0 |
| Squalane | 5.0 |
| Caprylic/capric triglyceride | 5.0 |
| Glycerin | 3.0 |
| Butylene glycol | 3.0 |
| Propylene glycol | 3.0 |
| Carboxyvinyl polymer | 0.1 |
| Triethanolamine | 0.2 |
| Preservatives, trace amounts of colorants, trace amounts of | 64.7 |
| fragrances, and trace amounts of purified water | |
| Total | 100.0 |

### Formulation Example 1-3: Nourishing Cream

A nourishing cream comprising a hybrid nanocomposite in which vegan exosomes and niosomes are fused according to the present disclosure as an active ingredient was prepared according to a conventional method as in Table 13 below.

TABLE 13

| Ingredient | Wt. % |
|---|---|
| Hybrid nanocomposite in which vegan exosomes and | 5.0 |
| niosomes are fused | |
| Beeswax | 10.0 |
| Polysorbate 60 | 1.5 |
| Sorbitan sesquioleate | 0.5 |
| Liquid paraffin | 10.0 |
| Squalane | 5.0 |
| Caprylic/capric triglyceride | 5.0 |
| Glycerin | 5.0 |
| Butylene glycol | 3.0 |
| Propylene glycol | 3.0 |
| Triethanolamine | 0.2 |
| Preservatives, trace amounts of colorants, trace amounts of | 51.8 |
| fragrances, and trace amounts of purified water | |
| Total | 100.0 |

### Formulation Example 1-4: Massage Cream

A massage cream comprising a hybrid nanocomposite in which vegan exosomes and niosomes are fused according to the present disclosure as an active ingredient was prepared according to a conventional method as in Table 14 below.

TABLE 14

| Ingredient | Wt. % |
|---|---|
| Hybrid nanocomposite in which vegan exosomes and | 5.0 |
| niosomes are fused | |
| Beeswax | 10.0 |
| Polysorbate 60 | 1.5 |
| Sorbitan sesquioleate | 0.8 |
| Liquid paraffin | 40.0 |
| Squalane | 5.0 |
| Caprylic/capric triglyceride | 4.0 |
| Glycerin | 5.0 |
| Butylene glycol | 3.0 |
| Propylene glycol | 3.0 |
| Triethanolamine | 0.2 |
| Preservatives, trace amounts of colorants, trace amounts of | 22.5 |
| fragrances, and trace amounts of purified water | |
| Total | 100.0 |

### Formulation Example 1-5: Pack

A pack comprising a vegan exosome-based hybrid according to the present disclosure as an active ingredient was prepared according to a conventional method as in Table 15 below.

TABLE 15

| Ingredient | Wt. % |
|---|---|
| Hybrid nanocomposite in which vegan exosomes and | 5.0 |
| niosomes are fused | |
| Polyvinyl alcohol | 13.0 |
| Sodium carboxymethyl cellulose | 0.2 |
| Allantoin | 0.1 |
| Ethanol | 5.0 |
| Nonylphenyl ether | 0.3 |
| Preservatives, trace amounts of colorants, trace amounts of | 76.4 |
| fragrances, and trace amounts of purified water | |
| Total | 100.0 |

### Formulation Example 2: Pharmaceutical Formulation

### Formulation Example 2-1: Preparation of a Gel

A gel was prepared by mixing a hybrid nanocomposite in which vegan exosomes and niosomes are fused according to the present disclosure with each ingredient of Table 16 below.

TABLE 16

| Ingredient | Wt. % |
|---|---|
| Hybrid nanocomposite in which vegan exosomes and | 5.0 |
| niosomes are fused | |
| Butylene glycol | 2.0 |
| Glycerin | 1.0 |
| Disodium EDTA | 0.02 |
| Sodium benzoate | 0.4 |
| Caprylic glycol | 0.2 |
| Sodium lactate | 0.5 |
| Hydroxyethyl cellulose | 0.7 |
| Purified water | 90.18 |
| Total content | 100.0 |

### Formulation Example 2-2: Preparation of an Ointment

An ointment was prepared by mixing a hybrid nanocomposite in which vegan exosomes and niosomes are fused according to the present disclosure with each ingredient of Table 17 below.

TABLE 17

| Ingredient | Wt. % |
|---|---|
| Hybrid nanocomposite in which vegan exosomes and | 5.0 |
| niosomes are fused | |
| Glyceryl monostearate | 1.0 |
| Sodium benzoate | 0.4 |
| Caprylyl glycol | 0.2 |
| Propylene glycol | 12.0 |
| Stearyl alcohol | 20.0 |
| White Vaseline | 25.0 |
| Purified water | 36.4 |
| Total content | 100.0 |

Although specific parts of the present disclosure have been described above in detail, it is apparent to those having ordinary skill in the art that such detailed descriptions are merely preferred embodiments, and that the scope of the present disclosure is not limited thereby. Accordingly, the substantial scope of the present disclosure will be defined by the appended claims and equivalents thereof.

1. A method for preparing a hybrid nanocomposite in which vegan exosomes and niosomes are fused, the method comprising:
(S10) preparing vegan exosomes by isolating exosomes from cells derived from plants or microorganisms, excluding animal cells;
(S20) inserting multiple bioactive substances into the vegan exosomes of step (S10);
(S30) preparing niosomes by mixing a non-ionic surfactant, a stabilizer, and a solvent, and performing high-pressure homogenization; and
(S40) fusing the vegan exosomes and the niosomes by ultrasonically treating the vegan exosomes into which the multiple bioactive substances are inserted in step (S20) and the niosomes of step (S30),
wherein the hybrid nanocomposite forms a bilayer or a trilayer by irregularly arranging a portion of a bilayer constituting the vegan exosomes and a portion of a bilayer constituting the niosomes, and the multiple bioactive substances inserted into the vegan exosomes are positioned on or attached to a surface of the hybrid nanocomposite, between membranes of the hybrid nanocomposite, or inside the hybrid nanocomposite.

2. The method of claim 1, wherein the bioactive substance is one or more selected from the group consisting of proteins, peptides, extracellular matrices, nucleic acids, lipids, carbohydrates, drugs, and phytochemicals.

3. The method of claim 1, wherein the insertion in step (S20) is performed by homogenizing a mixture of the vegan exosomes and the multiple bioactive substances at a pressure of 800 to 1200 bar using a homogenizer.

4. The method of claim 1, wherein the high-pressure homogenization in step (S30) is performed at 600 to 800 bar using a high-pressure homogenizer.

5. The method of claim 1, wherein the niosomes of step (S30) are additionally mixed with an auxiliary emulsifier.

6. The method of claim 1, wherein the mixing in step (S30) comprises 100 to 1000 parts by weight of the stabilizer and 180 to 220 parts by weight of the solvent, based on 100 parts by weight of the non-ionic surfactant.

7. The method of claim 1, wherein the vegan exosomes and the niosomes of step (S40) are mixed at a particle number of 0.1 to 2:0.1 to 2 and ultrasonically treated.

8. The method of claim 1, wherein the ultrasonic treatment in step (S40) is performed for 9 to 11 minutes using ultrasound of 30 to 50 kHz.

9. A hybrid nanocomposite in which vegan exosomes and niosomes are fused and into which the multiple bioactive substances are inserted, prepared by the preparation method of claim 1.

10. The hybrid nanocomposite of claim 9, wherein the vegan exosomes are exosomes isolated from cells derived from plants, plant callus, plant stem cells, plant undifferentiated cells, or microorganisms, excluding animal cells.

11. The hybrid nanocomposite of claim 9, wherein the bioactive substance is one or more selected from the group consisting of proteins, peptides, extracellular matrices, nucleic acids, lipids, carbohydrates, drugs, and phytochemicals.

12. A composition for delivering multiple bioactive substances comprising the hybrid nanocomposite in which vegan exosomes and niosomes are fused and into which the multiple bioactive substances are inserted, prepared by the preparation method of claim 1,
wherein the hybrid nanocomposite has an average particle diameter of 100 to 200 nm, forms a bilayer or a trilayer by irregularly arranging a portion of a bilayer constituting the vegan exosomes and a portion of a bilayer constituting the niosomes, and the multiple bioactive substances inserted into the vegan exosomes are positioned on or attached to a surface of the hybrid nanocomposite, between membranes of the hybrid nanocomposite, or inside the hybrid nanocomposite,
wherein the vegan exosomes are exosomes isolated from cells derived from plants or microorganisms, excluding animal cells, and
the bioactive substance is one or more selected from the group consisting of proteins, peptides, extracellular matrices, nucleic acids, lipids, carbohydrates, drugs, and phytochemicals.

13. A cosmetic composition comprising, as an active ingredient, the hybrid nanocomposite in which vegan exosomes and niosomes are fused and into which the multiple bioactive substances are inserted, prepared by the preparation method of claim 1,
wherein the hybrid nanocomposite has an average particle diameter of 100 to 200 nm, forms a bilayer or a trilayer by irregularly arranging a portion of a bilayer constituting the vegan exosomes and a portion of a bilayer constituting the niosomes, and the multiple bioactive substances inserted into the vegan exosomes are positioned on or attached to a surface of the hybrid nanocomposite, between membranes of the hybrid nanocomposite, or inside the hybrid nanocomposite,
wherein the vegan exosomes are exosomes isolated from cells derived from plants or microorganisms, excluding animal cells, and
the bioactive substance is one or more selected from the group consisting of proteins, peptides, extracellular matrices, nucleic acids, lipids, carbohydrates, drugs, and phytochemicals.

14. A topical composition for skin application, comprising, as an active ingredient, the hybrid nanocomposite in which vegan exosomes and niosomes are fused and into which the multiple bioactive substances are inserted, prepared by the preparation method of claim 1,
wherein the hybrid nanocomposite has an average particle diameter of 100 to 200 nm, forms a bilayer or a trilayer by irregularly arranging a portion of a bilayer constituting the vegan exosomes and a portion of a bilayer constituting the niosomes, and the multiple bioactive substances inserted into the vegan exosomes are positioned on or attached to a surface of the hybrid nanocomposite, between membranes of the hybrid nanocomposite, or inside the hybrid nanocomposite,
wherein the vegan exosomes are exosomes isolated from cells derived from plants or microorganisms, excluding animal cells, and
the bioactive substance is one or more selected from the group consisting of proteins, peptides, extracellular matrices, nucleic acids, lipids, carbohydrates, drugs, and phytochemicals.
